(19) United States
(12) Patent Application Publication
(10) Pub. No.: US 2018/0286519 A1
(43) Pub. Date: Oct. 4, 2018

(54) Methods and Systems for Extrapolating and Estimating Occurrences Based on Sample Data

(71) Applicant: Boston Strategic Partners, Inc., Boston, MA (US)

(72) Inventors: Friedrich William Peyerl, Centennial, CO (US); David Kazuo Hayashida, Boston, MA (US)

(21) Appl. No.: 15/943,318

(22) Filed: Apr. 2, 2018

Related U.S. Application Data
(60) Provisional application No. 62/479,789, filed on Mar. 31, 2017.

(51) Int. Cl. G16H 50/70; G16H 10/60; G06F 17/30

(52) U.S. Cl. CPC G16H 50/70; G06F 17/30598; G16H 10/60

(57) ABSTRACT

This disclosure relates to data analytics. The disclosed systems and methods allow for users to access one or more databases stored in a system. The systems and methods allow for real-time determination of outcomes for patients grouped into patient samples. The systems and methods allow for the grouping of patients into patient samples that can be analyzed by stratified hospital groups.

This application claims the benefit of priority of U.S. Provisional Application 62/479,789, which was filed on Mar. 31, 2017, the contents of which are incorporated in their entirety.

### FIELD

This disclosure relates generally to the field of data analytics.

### BACKGROUND

The World Health Organization published the International Classification of Diseases, ICD-6, in 1948 so as to harmonizing the classification of diseases and mortality for health studies and policy. However, the roots of the ICD date back to the International List of Causes of Death published by the International Statistical Institute in 1893 (see World Health Organization. International Classification of Diseases (ICD) Information Sheet at world wide web at who dot int/classifications/icd/factsheet/en/). Through the years, the ICD coding system has received major updates, including the approximately 17,000-code ICD-9 released in 1978, the more comprehensive 155,000-code ICD-10 in 1990, and a planned 2018 release named ICD-11. Today, over 100 nations use the ICD system, and jurisdictions such as the US have developed country-specific variants including ICD-9-CM, ICD-10-CM, and ICD-10-PCS, with their own adoption timelines (see CDC/National Center for Health Statistics. Classification of Diseases, Functioning, and Disability at world wide web at cdc dot gov/nchs/icd/index.htm).

Despite a large increase in comprehensiveness of coding with time, clinically differentiated diseases such as those that are refractory, rare, or poorly described are lost to a lack of coding specificity. These complex etiologies and pathophysiologies are often embedded within too-broadly defined ICD codes or are simply placed into codes with descriptions such as “unspecified” or “other.” Furthermore, there may be a lack of medical consensus over the clinical criteria for patients destined for a particular code. Be it a lack of classification, consensus, or combination thereof, the end result complicates the estimation of disease burdens, because these uncertainties have a cascading effect on the accuracy of downstream economic assessments.

One example of a disease which is poorly differentiated in the codes is refractory status epilepticus (RSE), a small subset of status epilepticus patients who experience life-threatening seizures that do not respond to traditional anti-convulsants. As a result, the epidemiologic features of RSE are poorly defined. Another example is hepatorenal syndrome (HRS) which is difficult to diagnose due to the rule-out nature of protocols. There is also a growing understanding of the effects of gender and underlying comorbidities which are not in the protocols today. With paperwork, discussion, and time, these under-recognized subsets may be granted dedicated codes thus increase disease awareness in the public sphere.

From the perspective of industry, weak or absent epidemiological data complicates prioritization of drug candidates and creation of marketing tools and pricing strategies. Particularly in rare diseases, inaccuracies in epidemiologic estimates can have large consequences in a company's ability to generate sufficient revenues to recover R&D sunk costs. To this end, drug companies may apply for an orphan drug designation in the US (and EU and other jurisdictions) under a government-run incentive program for rare indications (see Silverman E. Tiger in the Fiscal Room: Beware the Increasing Cost And Number of Orphan Drugs. Managed Care, March 2013). The orphan program helps companies reduce exposure to downside risk by granting extended patent protection, expedited review, and relaxed evidence requirements (see U.S. Food and Drug Administration. Designating an Orphan Product: Drugs and Biological Products).

However, an unrelenting rise in the number of drugs awarded orphan designation over the last 10 years has prompted questions from payers and hospitals over sustainability and value of these products against a paucity of epidemiological and economic evidence (see 6.U.S. Department of Health and Human Services, Food and Drug Administration, Center for Drug Evaluation and Research (CDER). Rare Diseases: Common Issues in Drug Development Guidance for Industry 11157dft.doc 07/29/15). A recent example is Sarepta Exondys 51, a USD300,000 per year drug indicated for Duchenne muscular dystrophy (DMD) which was approved by the FDA on the basis of a single, unpublished study of 13 patients. Referencing an additional two trials that did not meet their primary endpoints and FDA statements challenging the efficacy of the drug, Anthem, the 2nd largest insurance company in the US, has denied coverage of Exondys 51 (id.). More accurate discharge estimates are much-needed epidemiological tools that benefit multiple stakeholders.

Currently, the most commonly used epidemiology tool for inpatient hospital utilization is developed by the Healthcare Cost and Utilization Project (HCUP) National Inpatient Sample (NIS) (formerly Nationwide Inpatient Sample). The NIS is the largest publicly available all-payer hospital inpatient care database in the United States. It is a stratified systematic sample from State Inpatient Databases (SID) that cover 95% of US inpatient community hospital discharges. The NIS stratifies hospitals based on five characteristics: US Census Bureau division (US Census region prior to 2012), urban/rural location, teaching status, ownership, and bed size. These hospital strata are then assigned discharge weights, which are calculated by dividing the estimated number of universe discharges by the number of sampled discharges within each hospital stratum. Because of the systematic random sampling, the NIS can provide accurate national inpatient discharge estimates, specific to a calendar year, for various diagnosis/procedure codes.

The main limitation of the HCUP NIS public-facing web tools is the inability to define a complex cohort based on concurrent diagnosis and procedure ICD codes. HCUP also does not contain data such as clinical parameters, medication orders, or longitudinal histories—these are often critical to defining a more complicated illness and specialized subgroups. Furthermore, there are no computing means presently capable of organizing the massive amount of data available to obtain any determination of utilization or outcomes for complex patient samples.

### SUMMARY

Disclosed herein are systems and methods for the estimation of national discharges. The disclosed systems and methods produce a reliable epidemiology analysis of inpatient samples defined by clinical or coding criteria. The disclosed systems and methods can allow for individuals to access databases and the information stored in such databases through web-based applications. The systems and methods further allow for identifying a patient sample from a database stored on a computer using predefined clinical and/or coding criteria. Users can also input specific parameters to determine how the system categorizes a population. The systems and methods allow for stratification of hospitals and determination of discharge estimates for each stratum, as well as a national discharge estimate for a patient sample. The patient sample can thus represent an uncategorizable condition or other difficult to categorize characteristic of the patient sample that yields information regarding the discharge of the estimate. The discharge estimate can also determine the future outcome for the patient sample and costs of the patient sample.

The disclosed systems and methods include medical information. Such information includes pharmacy, laboratory, admission billing data from all patient care locations, data on patient demographics, encounters, diagnoses, prescriptions, procedures, laboratory test, locations of services/patients (e.g., clinic, ED, ICU, etc.) and hospital information.

Aspects disclosed herein include systems for modeling of the discharge rate of a patient sample. The systems can comprise at least one processor configured to access one or more computer-readable media, the media storing executable instructions to allow one or more users to access one or more databases stored on the computer-readable medium and generate a patient sample based on one or more patient characteristics. The methods can also comprise instructions to stratify a plurality of hospitals into strata based on one or more characteristics of the plurality of hospitals, to determine a ratio of inpatient discharges based on information stored in a database, and to calculate a discharge weight for each strata. In certain embodiments, the systems comprise instructions to calculate an estimate for each strata, the estimate being calculated from the patient sample, the ratio of inpatient discharges, and the discharge weight and determine an aggregate national discharge estimate based on the estimate for each strata.

In some embodiments, the patient sample is grouped by a characteristic selected from the group consisting of disease, age, weight, Medicaid status, Medicare status, income level, treatment regimen, predicted outcome, comorbidities, and combinations thereof. In other embodiments, the patient sample is grouped according to orphan drug status.

In certain embodiments, the system comprises instructions allowing the one or more users to access the system over a network through a web browser. In particular embodiments, the inpatient discharge ratio is the ratio of HCUP inpatient discharges to Cerner inpatient discharges.

In still other embodiments, the system comprises instructions to determine the future treatment cost of the patient sample by taking the aggregate national estimate and comparing the national estimate to other like patient samples stored in the computer-readable medium. In still further embodiments, the system comprises instructions for creating the patient sample depending on one or more characteristics selected from the group consisting of illness, illness severity, outcomes of treatments, treatment regimens, age, hospital, location, Medicaid status, Medicare status, comorbidities, and combinations thereof.

In some embodiments, the discharge weight is calculated from discharge rates for all hospitals in a stratum and an adjusted discharge rate. In yet more embodiments, the adjusted discharge rate is calculated as a product of inpatient discharge days and one plus the ratio of gross outpatient revenue to gross inpatient revenue.

In certain embodiments, hospitals are stratified by one or more characteristics selected from the group consisting of number of beds, specialties, teaching hospital status, number of doctors, regional location, urban/rural status, and performance metrics.

Aspects discloses herein involve methods of determining an outcome for a patient sample. The methods can include identifying a patient sample according to one or more characteristics relevant to members of the patient sample, identifying one or more hospital groups based on one or more characteristics relevant to each member of the group, segregating each hospital group into one or more strata, and determining a ratio between two discharge known discharge rates for each strata.

In some embodiments, the methods comprise determining a discharge weight for each strata, calculating a discharge estimate for each strata based on the patient sample, the ratio, and the discharge weight, and aggregating the discharge estimates of each strata to identify a national discharge estimate. In other embodiments, the national discharge estimate provides an estimate of outcome for a patient sample.

In certain embodiments, the patient sample is grouped by a characteristic selected from the group consisting of disease, age, weight, Medicaid status, Medicare status, income level, treatment regimen, predicted outcome, comorbidities, and combinations thereof. In particular embodiments, the patient sample is grouped according to orphan drug status. In more particular embodiments, the patient sample is grouped according to treatment regimen.

In some embodiments, the discharge ratio is the ratio of HCUP inpatient discharges to Cerner inpatient discharges. In other embodiments, the method comprises determining the future treatment cost of the patient sample by taking the aggregate national estimate and comparing the national estimate to other like patient samples stored in the computer-readable medium. In still other embodiments, the method comprises creating the patient sample depending on one or more characteristics selected from the group consisting of illness, illness severity, outcomes of treatments, treatment regimens, age, hospital, location, Medicaid status, Medicare status, comorbidities, and combinations thereof.

In certain embodiments, the discharge weight is determined by averaging the discharge rates for all hospitals in a stratum and an adjusted discharge rate. In more certain embodiments, the adjusted discharge rate is calculated as a product of inpatient discharge days and one plus the ratio of gross outpatient revenue to gross inpatient revenue.

In further embodiments, hospitals are stratified by one or more characteristics selected from the group consisting of number of beds, specialties, teaching hospital status, number of doctors, regional location, urban/rural status, and performance metrics.

### BRIEF DESCRIPTION OF THE FIGURES

To further understand the systems and methods disclosed herein, reference is directed to the following brief description of the figures as well as the drawings that show exemplary embodiments of the systems and methods:

FIG. 1 is a schematic representation showing an embodiment of a system.

FIG. 2 is a schematic representation showing an embodiment of instructions or modules of instructions that the system of FIG. 1 utilizes.

FIG. 3 is a schematic representation showing how the system determines national estimates.

FIG. 4 shows a representation of how an embodiment of the systems determines outcomes and costs.

### DETAILED DESCRIPTION

Disclosed herein are systems that allow for estimation of national discharges. The disclosed systems allow for national estimates of national inpatient visit estimates for all available ICD-9 codes in the Cerner Health Facts® database. Cerner Health Facts® is a proprietary, US-only electronic health records (EHR) database of ˜63 million patients from year 2000 to 2016, representing approximately 10-20% of all health care encounters in US (see world wide web at cerner dot com/uploadedFiles/f103_461_09_v4_health_facts_researchers.lr.pdf Accessed Dec. 13, 2016). The disclosed systems further allow for one-to-one comparison against all available ICD-9 diagnosis code national estimates from HCUP.

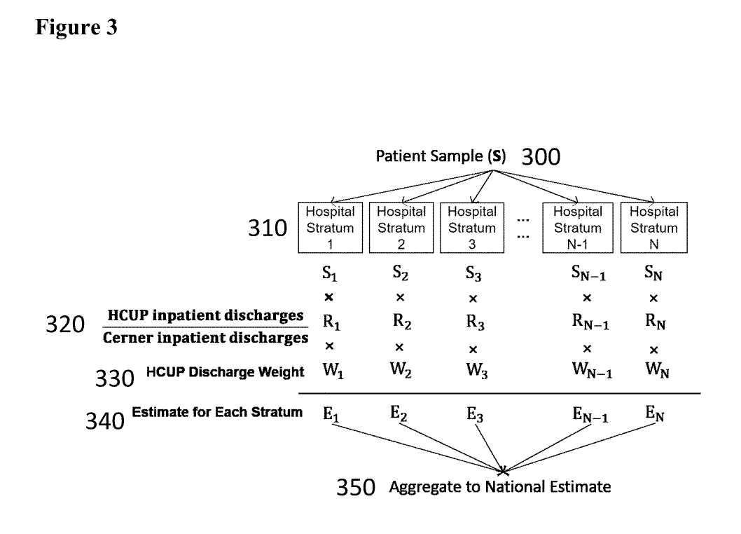

Aspects of the methods and systems disclosed herein include identifying a patient sample from a database using predefined clinical and/or coding criteria. The methods further include stratifying characteristics, such as hospital characteristics. In some embodiments, the characteristic comprises hospital demographics such that the system identifies a hospital match in a database during the extrapolation process. In some embodiments, the methods further comprises calculating a ratio of inpatient visits to a pre-determined equivalent cohort in the database. The pre-determined equivalent cohort can be within matching hospital strata by year of service and the NIS stratification variables—US Census Bureau division (US Census region prior to 2012), urban/rural location, teaching status, ownership, and bed size. In some embodiments, once the ratio is determined for each strata, the ratios are multiplied by particular discharge weights for each respective stratum, the result of which is then summed to yield a nationwide discharge projection. FIG. 3 shows the steps taken by the system to perform the stratification.

The disclosed systems comprise a memory operably linked to a processor such that the processor can access and execute instructions stored on the memory. In certain embodiments, the instructions are modules that the processor accesses and executes. For example, the processor can access a module of instructions that allows the system to perform a particular task. In some embodiments, the system comprises instructions that allow the system to receive data relating to a one or more codes, one or more hospital characteristics, and/or one or more patient characteristics. In other embodiments, the system can comprise instructions for the system to store data relating to the codes, discharges, patient characteristics, and/or hospital characteristics. In still other embodiments, the system comprises instructions to pre-determine an equivalence between cohorts.

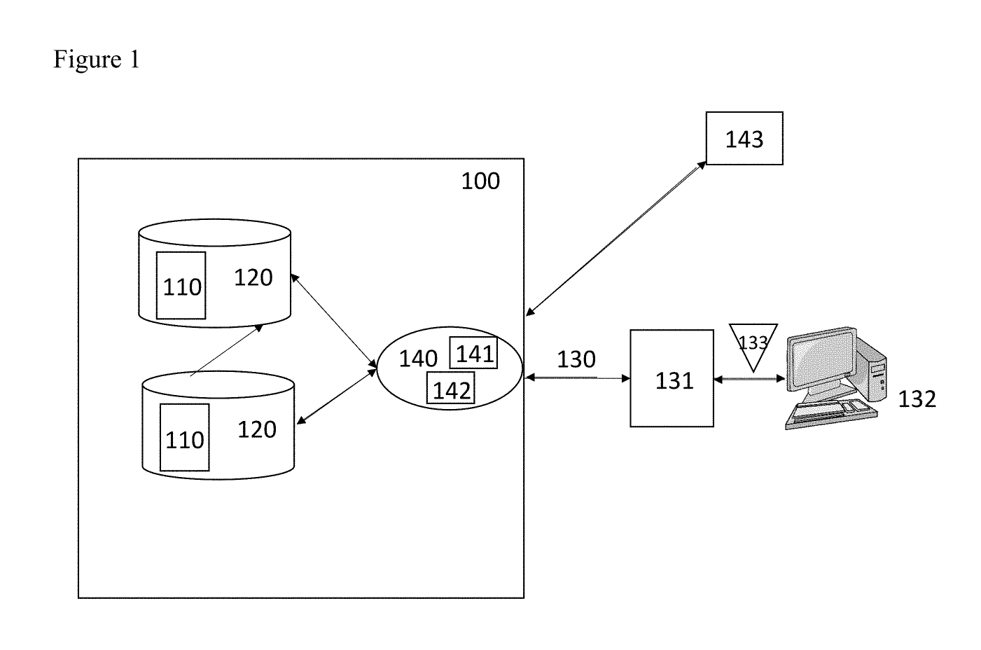

FIG. 1 shows an embodiment of the disclosed systems. The system 100 comprises a database 110. The database 110 is stored in a computer-readable storage medium 120. The computer-readable storage medium can exist as an off-line storage medium, as well as primary, secondary, or tertiary storage mediums. Examples of computer-readable storage medium include CD-ROM, CD-RW, DVD-RW, DDS, hard disk drive, mass storage device, removable media drive, and robotic storage libraries.

The system 100 further comprises a connection 130 to a network 131. The network 131 allows for a user 132 to access the system 100. The user 132 can access the network 131 through a web browser (not shown). The user 132 can access the system 100 through a web application 133 that allows the user 132 to provide inputs to the system 100.

In certain embodiments, the web application 133 prompts the user 132 to provide information relating to a year to be analyzed, hospital information such as numbers of beds, rural/urban location, teaching status, ownership, and other information that can be obtained from the U.S. Census or other databases. The information can also be related to a patient sample, including ICD codes in databases such as Health Facts® and HCUP. The user 132 can also input information relating to discharges in a patient sample in one or more hospitals. The web application 133 allows for the user 132 to receive information from the system 100 relating to an estimate for a particular hospital stratum and/or a calculated national estimate of all strata of hospitals. The information sent from the system can be accessed as an HTML, a PDF, a JPEG, or other electronic document

Furthermore, the system can comprise security to ensure that only authorized users can access sensitive data. Encryption techniques are known to those of ordinary skill in the art. Examples of encryption include Sophos SafeGuard Enterprise Encryption 7 and Vormetric Transparent Encryption.

The system 100 may comprise one or more servers 140, each of which comprise a processor 141 and memory 142. The servers 140 provide a network interface between the user 132 and the system 100. In addition, network interfaces can be established between devices within the system. The servers 140 also provide a network interface between other computers 143 in the system 100 and the computer-readable storage medium 120. The system 100 may also comprise one or more computers 143, each of which can also comprise a processor and memory.

As shown in FIG. 1, the system 100 comprises executable instructions located on the servers 140 in the memory 120. The instructions can be modules of instructions that allow the system to perform certain tasks. Regarding the operation of the system 100, the processor 141 executes the instructions.

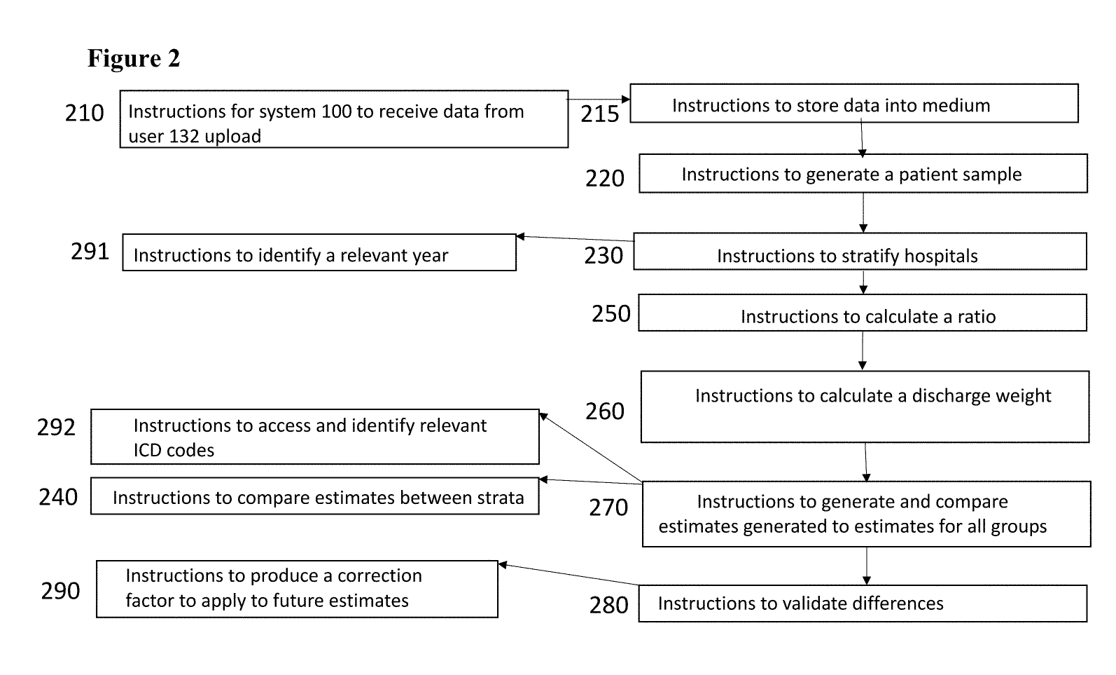

FIG. 2 shows the instructions that the system 100 of FIG. 1 comprises. The system 100 comprises instructions 210 that allow the system 100 to receive information (e.g., data) relating to user 132 requests. The instructions 210 allow a user 132 to upload or input information into a database in the system 100. The instructions 210 also allow the user 132 to upload requests into the system 100 for analysis. The database 110 can be continually updated with information inputted from users 132 relating to hospital strata or to discharge information for particular hospital strata.

The system 100 further comprises instructions 215 that allow the system to store uploaded or inputted information into a computer-readable medium 120. The stored data can be used to generate models and/or estimates relating to an estimate of cost of a particular disease. The system 100 further comprises instructions to generate a patient sample. The patient sample can be generated according to one or more characteristics, including disease state, age, drug regimen, race, gender, or combinations thereof. The system 100 groups the patient sample according to one or more of the characteristics and calculates the number of patients having a particular disease condition of interest within the sample. These comparisons are performed using instructions that allow for the system to identify patient records in which similarities in a field are identified and patient records are binned together within a sample. The system 100 also comprises instructions 240 that allow the system to compare estimates from different hospital strata. The comparisons are performed by calculating at least two estimates for two strata. Each estimate is compared to determine the differences between the strata. For instance, the system 100 can identify each estimate for each strata and compares them using tables where each estimate is compared to one other value until all estimates have been compared to one another. The system 100 comprises instructions in which the system 100 determines whether either of two values of a comparison are equal within a range of error, one value is greater than the other value, or whether the other value is greater than the one value. The system 100 can thus determine which estimates are equal to one another and group the estimates.

The system 100 comprises instructions 230 that allow the system to stratify hospitals so as to calculate an estimate for one or more hospital strata. The system 100 can stratify hospitals by one or more characteristics selected from the group consisting of number of beds, specialties, teaching hospital status, number of doctors, regional location, urban/rural status, and performance metrics.

The system 100 also comprises instructions 250 that allow for the calculation of a ratio relating to inpatient discharges. In some embodiments, the ratio is calculated based on discharges in the HCUP system and the Cerner Health Facts® system. The ratio can also be based on discharge information obtained for one or more hospitals. In other embodiments, the ratio is calculated from inpatient discharge values provided by the user. The ratio can be calculated by taking the quotient of division of the HCUP system and the Cerner system.

Additional aspects of the system 100 comprises instructions 260 to calculate a discharge weight for each hospital stratum. The discharge weight can be obtained from available sources such as HCUP (see world wide web at hcup-us dot ahrq.gov/db/nation/nis/trendwghts.jsp). In particular embodiments, the discharge weight is calculated by the system 100 based on information received from the user. For instance, the system 100 receives an input from a user instructing the system 100 to change the discharge weight to a particular value. In still other embodiments, the system 100 calculates a new weighted average based on information received and stored in computer-readable storage medium 120. The system 100 generates discharge weights by determining the ratio of discharges in all hospitals to the discharges in the sample. In particular embodiments, the system 100 calculates a discharge weight by determining the ratio of discharges in all hospitals to the discharges in the sample within a particular category of patient such as Medicare, Medicaid, newborns, children, and adult categories.

The system 100 can calculate a discharge weight using the following equation:

W(all hospitals in stratum)=[N(discharges all hospitals in stratum)/×N(adjusted discharges)]*(4/Q)

Where Q is the number of quarters of discharge data provided by a hospital to a database. In some embodiments, the system 100 receives the data for Q.

The system 100 is configured to generate a national estimate for a patient sample. The system 100 calculates the national estimate by determining an aggregate estimate for all strata. The aggregated estimate is calculated by summing the estimates for each strata and dividing by the total number of strata. FIG. 3 shows an embodiment of the analysis performed by the system 100. As shown in FIG. 3, the system 100 receives data for a patient sample (S) 300 in hospitals. The hospitals are placed into strata 310 based on the hospital characteristics. Examples of characteristics that are used to place a hospital into a stratum include number of beds, the location of the hospital, urban/rural, specialty, region of the hospital, number of medical professionals, teaching status, or combinations thereof.

In FIG. 3, the system 100 further determines the ratio of HCUP inpatient discharges to Cerner inpatient discharges 320. The system 100 also determines a discharge weight 330 to apply to each stratum. FIG. 3 shows that the discharge weight 330 is the HCUP discharge weight. In other embodiments, the discharge weight is calculated by the system 100. In still other embodiments, the discharge weight 330 is received by the system 100 from a user. The system 100 further calculates the estimate for each stratum 340 and the aggregate national estimate 350.

Returning to FIG. 2, the system 100 has instructions 270 to compare the estimates generated to estimates for all available all groups of patients and in some instances to all ICD diagnosis code prevalence estimates. In some embodiments, the estimates are obtained from HCUP. In other embodiments, the system 100 receives estimates from a user. In particular embodiments, the estimates are stored in a database stored in the computer-readable storage medium 120. In still other embodiments, the system 100 generates a null hypothesis that there is no difference between the system generated estimates and the estimates for all available ICD diagnosis code prevalence estimates.

The system 100 in exemplary embodiments further comprises instructions 280 to validate the difference between system generated estimates and estimates available for the patient groups. The groups are validated by OLS linear regression models to quantify the relationship of the extrapolation to the estimates. In some embodiments, the system 100 also comprises instructions 290 to generate a correction factor that the system 100 applies to future system generated estimates and estimates available for all patient groups.

In particular embodiments, the system 100 comprises instructions 291 to identify a year for determining estimates for hospital strata and national aggregation estimates. In some embodiments, the year selected by the system 100 is the most year in which data is obtained. In some embodiments, the system 100 stores in a memory 120 all available diagnosis code discharges for the calendar year from one or more databases. In certain embodiments, the system 100 comprises instructions 292 to access one or more codes stored in memory 120. For instance, there are 9,831 pair-able ICD-9 codes prior to the selection of diseases with >10 discharges. In some embodiments, the system 100 removes all codes in which there are less than or equal to 10 discharges.

Aspects of the disclosed system 100 comprises instructions 294 to perform a correlation check (SAS, PROC CORR) between the system estimate and the difference of the system estimate to a reference estimate (such as a HCUP estimate). For example, the correlation check establishes a threshold below which an insufficient n would introduce bias into the difference between estimates for any individual code. After discarding low n codes, the system 100 determines the mean difference when a deviation of +/−5% or less was achieved.

In some embodiments, the system 100 performs a subgroup analysis. A subgroup analysis is performed to identify differences between system estimates and estimates for all available ICD diagnostic codes. For instance, through subgroup analysis, an approximate threshold of discharges can occur below which the count of the discharges appear to affect the magnitude of the difference in estimates. In more particular embodiments, the system 100 determines a convergence to a mean difference in discharge estimates.

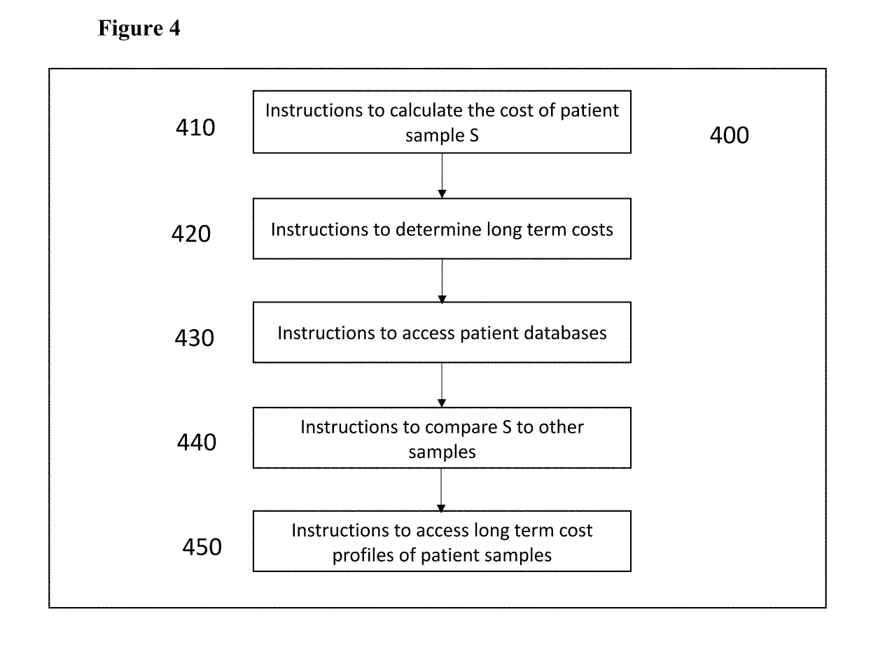

Aspects of the disclosed systems comprise instructions to allow for determination of the cost associated with a patient sample. In some embodiments, the determination of cost is by determining cost data in a source and using the weighted discharge data to calculate a national estimate or an estimate for each stratum. As shown in FIG. 4, the system 400 comprises instructions 410 to determine the costs of a patient sample. The determination involves the determined national discharge estimate determined by the system for the patient sample. The system 400 determines the likely cost of treating the patient sample based on the discharge estimate. For instance, if the national discharge estimate for the patient sample is determined to be 30 days, the system 400 can determine the likely costs for such a patient sample by comparing the patient sample to other patient samples having similar discharge estimates and having determined cost estimates. In other embodiments, the system 400 calculates the costs of based on parameters such as cost of daily inpatient care in the nation.

In some embodiments, the system 400 comprises instructions 420 to determine the long term cost of a patient sample based on discharge estimates and attributes of the patient sample. For instance, the system 400 comprises instructions 430 to access patient sample databases relating to disease, ICD code, age, BMI, behavioral attributes, and other medically relevant information to the patient sample. The system 400 comprises instructions 440 to compare the attributes of the patient sample to attributes of other patient samples having known long term cost profiles. In some embodiments, the system 400 comprises instructions 450 to access long term cost profiles for particular attributes.

Aspects of the disclosed herein relate to methods of categorizing and identifying discharge outcomes for patient samples. In particular embodiments, the methods involve identifying a patient sample. Examples of patient sample categorization include grouping patients according to disease, ICD code, treatment regimen, age, weight, regional location, hospital location, wealth status, Medicaid status, Medicare status, or other medically relevant information about patients being grouped. The patient sample can be grouped according to any one or more of the characteristics. In particular embodiments, each member of the patient sample has at least one characteristic of the other members of the group. The patient sample can also be grouped according to orphan disease status or treatment regimen.

The methods disclosed herein include identifying and grouping hospitals into strata. Hospital can be stratified by one or more characteristics selected from the group consisting of number of beds, specialties, teaching hospital status, number of doctors, regional location, urban/rural status, and performance metrics. Hospitals can also be stratified according to urban, suburban, or rural location, association with medical schools, admission rates, discharge rates, billing information, and readmission rates. Once one or more hospital strata are formed, the patient samples are associated with each hospital strata.

The methods also include determining a ratio between at least two discharge rates. For instance, the discharge ratio can be the ratio of HCUP inpatient discharges to Cerner inpatient discharges. In other embodiments, the system calculates a discharge ratio based on information stored in the computer-readable medium to determine a ratio for each hospital stratum.

Aspects of the methods comprise the calculation of a discharge weight for each stratum.

As noted herein, the discharge weight can be determined using the formula

W(all hospitals in stratum)=[N(discharges all hospitals in stratum)/×N(adjusted discharges)]*(4/Q)

Where Q is the number of quarters of discharge data provided by a hospital to a database. In some embodiments, the system 100 receives the data for Q.

In some embodiments, the methods comprise determining the future treatment cost of the patient sample by taking the aggregate national estimate and comparing the national estimate to other like patient samples stored in the computer-readable medium. Such a determination allows for healthcare providers and insurers to identify the costs associated with diseases, even those diseases that are hard to categorize. As disclosed herein, the methods allow for comparison of a patient sample to diseases having well-known outcomes and costs.

In certain embodiments, the methods comprise identifying a discharge weight. The discharge weight can be determined by averaging the discharge rates for all hospitals in a stratum and an adjusted discharge rate. In more certain embodiments, the adjusted discharge rate is calculated as a product of inpatient discharge days and one plus the ratio of gross outpatient revenue to gross inpatient revenue.

In some embodiments, the methods comprise determining an estimate for each stratum by multiplying the patient sample in each stratum to the determined ratio and then multiplying this product to a determined discharge weight. The patient sample is the incidence or prevalence of the disease in the source sample. The value that is determined by the operation is the number is an estimate for each stratum. These values are aggregated to determine the national estimate for the patient sample.

Aspects of the disclosed methods allow users to access a system in which information relating to patient samples and hospitals are stored. The methods comprise storing information relating to a plurality of patient samples in a database in a computer-readable storage medium located on a storage device. In some embodiments, the storage device is located on a server or computer. The user can upload information relating to a specimen or population of specimens for storage. In some embodiments, the system allows one or more users to enter information into a database relating to a poorly characterized patient population to create a new patient sample. In particular embodiments, the system compares the patient sample to other known cohorts.

The methods further can comprise pre-determining at least one characteristic for the patient sample stored in the database. In certain embodiments, the methods comprise categorizing the patient sample. In particular embodiments, the patient sample is categorized based on information that the user inputs into the system. The information can include characteristics of the patient sample. Exemplary characteristics include illness, illness severity, outcomes of treatments, age, Medicaid status, Medicare status, hospital location, and comorbidities. The characteristics can include Elixhauser and Charlson comorbidity indices, and hospital characteristics.

Aspects of the methods disclosed herein also comprise accessing a storage device. The storage device can be accessed through a network interface when the storage device is connected to one or more servers through a network connection. In certain embodiments, the system accesses a database through a network interface.

### EQUIVALENTS

Those skilled in the art will recognize, or be able to ascertain, using no more than routine experimentation, numerous equivalents to the specific embodiments described specifically in this disclosure. Such equivalents are intended to be encompassed in the scope of the following claims.

What is claimed:

1. A system for real-time modeling of the discharge rate of a patient sample comprising:
at least one processor configured to access one or more computer-readable media, the media storing executable instructions to:
i) allow one or more users to access one or more databases stored on the computer-readable medium;
ii) generate a patient sample based on one or more patient characteristics;
iii) stratify a plurality of hospitals into strata based on one or more characteristics of the plurality of hospitals;
iv) determine a ratio of inpatient discharges based on information stored in a database;
v) calculate a discharge weight for each strata;
vi) calculate an estimate for each strata, the estimate being calculated from the patient sample, the ratio of inpatient discharges, and the discharge weight; and
vii) determine an aggregate national discharge estimate based on the estimate for each strata.

2. The system of claim 1, wherein the patient sample is grouped by a characteristic selected from the group consisting of disease, age, weight, Medicaid status, Medicare status, income level, treatment regimen, predicted outcome, comorbidities, and combinations thereof.

3. The system of claim 1, wherein the patient sample is grouped according to orphan drug status.

4. The system of claim 1 further comprising instructions allowing the one or more users to access the system over a network through a web browser.

5. The system of claim 1, wherein the inpatient discharge ratio is the ratio of HCUP inpatient discharges to Cerner inpatient discharges.

6. The system of claim 1 further comprising instructions to determine the future treatment cost of the patient sample by taking the aggregate national estimate and comparing the national estimate to other like patient samples stored in the computer-readable medium.

7. The system of claim 1 further comprising creating the patient sample depending on one or more characteristics selected from the group consisting of illness, illness severity, outcomes of treatments, treatment regimens, age, hospital, location, Medicaid status, Medicare status, comorbidities, and combinations thereof.

8. The system of claim 1, wherein the discharge weight is calculated from discharge rates for all hospitals in a stratum and an adjusted discharge rate.

9. The system of claim 8, wherein the adjusted discharge rate is calculated as a product of inpatient discharge days and one plus the ratio of gross outpatient revenue to gross inpatient revenue.

10. The system of claim 1, wherein hospitals are stratified by one or more characteristics selected from the group consisting of number of beds, specialties, teaching hospital status, number of doctors, regional location, urban/rural status, and performance metrics.

11. A method of determining an outcome for a patient sample, the method comprising:
a) providing a processor operably connected to one or more computer-readable memories;
b) accessing one or more databases stored in the computer-readable memories;
b) identifying in the one or more databases a patient sample according to one or more characteristics relevant to members of the patient sample;
c) identifying in the one or more databases one or more hospital groups based on one or more characteristics relevant to each member of the group;
d) segregating each hospital group into one or more strata;
e) determining a ratio between two discharge known discharge rates for each strata;
f) determining a discharge weight for each strata;
g) calculating a discharge estimate for each strata based on the patient sample, the ratio, and the discharge weight; and
h) aggregating the discharge estimates of each strata to identify a national discharge estimate,
wherein the national discharge estimate provides an estimate of outcome for a patient sample.

12. The method of claim 11, wherein the patient sample is grouped by a characteristic selected from the group consisting of disease, age, weight, Medicaid status, Medicare status, income level, treatment regimen, predicted outcome, comorbidities, and combinations thereof.

13. The method of claim 11, wherein the patient sample is grouped according to orphan drug status.

14. The method of claim 11, wherein the patient sample is grouped according to treatment regimen.

15. The method of claim 11, wherein the discharge ratio is the ratio of HCUP inpatient discharges to Cerner inpatient discharges.

16. The method of claim 11 further comprising determining the future treatment cost of the patient sample by taking the aggregate national estimate and comparing the national estimate to other like patient samples stored in the computer-readable medium.

17. The method of claim 11 further comprising creating the patient sample depending on one or more characteristics selected from the group consisting of illness, illness severity, outcomes of treatments, treatment regimens, age, hospital, location, Medicaid status, Medicare status, comorbidities, and combinations thereof.

18. The method of claim 11, wherein the discharge weight is determined by averaging the discharge rates for all hospitals in a stratum and an adjusted discharge rate.

19. The method of claim 18, wherein the adjusted discharge rate is calculated as a product of inpatient discharge days and one plus the ratio of gross outpatient revenue to gross inpatient revenue.

20. The method of claim 11, wherein hospitals are stratified by one or more characteristics selected from the group consisting of number of beds, specialties, teaching hospital status, number of doctors, regional location, urban/rural status, and performance metrics.
